(19) United States
(12) Patent Application Publication
(10) Pub. No.: US 2005/0005324 A1
(43) Pub. Date: Jan. 6, 2005

(54) Gene promoters isolated from potato and use thereof

(76) Inventors: Ziyu Dai, Richland, WA (US); LiFang Shi, Richland, WA (US); Brian S. Hooker, Kennewick, WA (US)

Correspondence Address: WELLS ST. JOHN P.S., 601 W. FIRST AVENUE, SUITE 1300, SPOKANE, WA, 99201, US

(21) Appl. No.: 10/903,948

(22) Filed: Jul. 30, 2004

Related U.S. Application Data
(62) Division of application No. 10/051,307, filed on Jan. 22, 2002.
(60) Provisional application No. 60/263,224, filed on Jan. 23, 2001.

(51) Int. Cl. C12N 15/82; C07H 21/04; C12N 15/87; C12N 15/09; C12N 15/63; C12N 5/10

(52) U.S. Cl. 800/278; 536/24.1; 536/23.6; 435/320.1; 435/419; 435/468

(57) ABSTRACT

There are disclosed novel pin1 gene promoter isoforms and amt gene promoter isoforms. The promoters and their functional elements may be used independently or in combination with one or more enhancer elements to increase or otherwise manipulate gene expression. The promoters disclosed may be used in Controlled Environment Agriculture for heterologous protein production. Also disclosed are methods for using the promoter and promoter elements of the instant invention, as well as vectors and transgenic plants comprising the same.

### BACKGROUND OF THE INVENTION

1. Field of the Invention

The present invention relates generally to genetic transcription control mechanisms in plants. More specifically, the present invention relates to gene expression in transgenic plants. Most particularly, the present invention relates to isoforms of the proteinase inhibitor 1 (pin1) promoter and the aminotransferase (amt) promoters and their use in manipulating expression of genes, especially in transformed plant cells.

2. Description of the Related Art

Given the technological advances in recombinant DNA technology made over the past decade it has become common practice to introduce new genetic material into plant cells, plant tissues or a whole plant to establish new traits that enhance the value of the plant or plant tissues. Both angiosperm and gymnosperm higher plants are included within the definition of “plant.”

A typical eukaroytic gene consists of a promoter region, introns, exons and a transcription terminator. The promoter region is typically located upstream of the transcribed region. The promoter determines the level and specificity of gene transcription.

In eukaryotic organisms such as a plant, a promoter is not recognized directly by a RNA polymerase. Transcription initiation factors (TIFs) first bind to a promoter to form a preinitiation complex, and only then does an RNA polymerase bind to form an initiation complex.

A promoter for RNA polymerase II consists of a transcription initiation region, generally including a TATA box (the “Goldberg-Hogness Box”) and frequently a CCAAT box, as well as upstream cis-acting elements. The transcription initiation region is also called the minimal promoter because it is the minimal DNA sequence required for gene transcription. The TATA box directly binds a transcription factor complex that includes RNA polymerase II, for the initiation of DNA transcription. The TATA box is located approximately 25 base pairs upstream of the transcription start site. Further upstream, often between nucleotides −80 and −100, there can be a promoter element with homology to the consensus sequence CCAAT. Breathnach et al., Ann. Rev. Biochem. 50:349-383 (1981). In plants, the CCAAT box may be substituted by the AGGA box, at a similar distance from the transcription start site. Messing et al., in Genetic Engineering of Plants, Kosuge et al. Eds., pages. 211-227.

Promoters, together with enhancers and silencers, are cis-acting elements that control gene expression. Promoters are positioned next to the transcription start site and function in an orientation-dependent manner. Enhancer and silencer elements, which modulate the activity of promoters, may affect promoter activity in either orientation and at greater distances from the transcription start site. Khoury et al., Cell 33:3-13 (1983).

Enhancers can greatly increase the rate of transcription, and can generally function in either orientation and at various distances upstream or downstream from a given promoter. Enhancers may function in a wide variety of cells, or they may show a preference for expression in certain cells or tissues. Enhancers may affect gene expression in response to environmental stimuli, such as illumination, nutrient concentration, heat shock, wounding, and anaerobiosis. These elements may also control gene expression in a development-preferred or tissue-preferred manner.

Typically, enhanced mRNA expression is desired to increase the level of expression of the protein encoded by this mRNA. In addition, development-preferred expression patterns enable protein production in plants during desired developmental stages, for example, post-harvest synthesis of foreign proteins. Also, tissue-preferred patterns of expression enable novel schemes for utilization of non-crop plant parts for protein production as well as conferring necessary traits, such as disease resistance or chemical tolerance, to preferred tissues. As recombinant DNA techniques are increasingly being applied to higher plants, there is an increased need for novel promoter elements to enable artificial regulation of gene expression. Specifically, there is a need for novel promoter elements that enable high levels of expression that are temporally, environmentally or developmentally regulatable. In addition, where multiple genes are controlled by a single promoter, suppression of expression may result. There is a need, therefore, for new and different promoters in plants to regulate stacked traits.

### SUMMARY OF THE INVENTION

It is therefore an object of the present invention to provide novel promoter elements that enable high level gene expression and flexible control of such expression. It is another object of the present invention to provide a method for increased gene expression at high levels in a temporally, environmentally or developmentally controlled manner.

In a first embodiment, the invention provides an isolated polynucleotide having at least 70% sequence identity with the nucleotide sequence shown in FIG. 1 and pin1 gene promoter activity.

In another embodiment, the invention provides an isolated DNA sequence comprising a polynucleotide molecule selected from the group consisting of that shown in FIG. 1, FIG. 2, FIG. 3, and any functional elements thereof having pin1 promoter activity.

In another embodiment, the invention provides an isolated polynucleotide having at least 70% sequence identity with the nucleotide sequence shown in FIG. 4 and amt gene promoter activity.

In another embodiment, the invention provides an isolated DNA sequence comprising a polynucleotide molecule selected from the group consisting of that shown in FIG. 4, FIG. 5, and functional elements thereof having pin1 promoter activity.

In another embodiment, the invention provides a cDNA molecule having the nucleotide sequence shown in FIG. 8 which corresponds to the amt1 gene.

In yet another embodiment, the invention provides a cDNA molecule having the nucleotide sequence shown in FIG. 9 which corresponds to the amt gene.

Further embodiments of the invention provide a recombinant expression vector comprising the promoter or promoter elements, a plant cell comprising the expression vector, a transgenic plant regenerated from the cell, and a method for producing a protein of interest in transgenic plants by means of operably linking a promoter of the present invention to a gene coding for the proteins. The promoters of the present invention may be used in Controlled Environment Agriculture (CEA) to make heterologous proteins of interest.

### BRIEF DESCRIPTION OF THE DRAWINGS

FIG. 1. illustrates the DNA sequence of isoform 1 of the pin1 gene promoter highlighting the translation start site, TATA box and CAATT box.

FIG. 2. illustrates the DNA sequence of isoform 2 of the pin1 gene promoter highlighting the translation start site, TATA box and CAATT box.

FIG. 3. illustrates the DNA sequence of isoform 3 of the pin1 gene promoter highlighting the translation start site, TATA box and CAATT box.

FIG. 4. illustrates the DNA sequence of isoform 1 from amt gene promoter highlighting the translation start site, TATA box and CAATT box.

FIG. 5. illustrates the DNA sequence of isoform 1 from amt gene promoter highlighting the translation start site, TATA box and CAATT box.

FIG. 6. shows the results of Northern blot analysis for pin1 gene expression in a transformed plant incubated in the dark.

FIG. 7. Shows the results of a Northern blot analysis for amt gene expression in a transformed plant incubated in the dark.

FIG. 8. illustrates the DNA sequence of the cDNA corresponding to the pin1 gene.

FIG. 9. illustrates the DNA sequence of the cDNA corresponding to the amt gene.

FIG. 10. shows the results of a Northern blot analysis for pin1 expression in a transformed plant exposed to treatment with ethylene.

FIG. 11. Shows the results of a Northern Blot analysis for amt gene expression in a transformed plant exposed to treatment with ethylene.

### DETAILED DESCRIPTION OF THE PREFERRED EMBODIMENT

The present invention provides novel promoter isoforms that enable high level gene expression and flexible control of such expression. The present invention also provides a method for increased gene expression at high levels in a temporally, environmentally or developmentally controlled manner.

The promoters may induce high level, stable, and controllable expression of an operably linked gene. The various promoter isoforms and their elements according to the present invention may be combined with a plurality of other promoter elements to provide for enhanced gene expression and increased control of gene expression via environmental and developmental parameters. The promoters and promoter elements according to the present invention are particularly suitable for enhanced gene expression and regulation of transcription of plant genes.

1. Definitions

A structural gene is a DNA sequence that is transcribed into messenger RNA (mRNA). The mRNA may be translated into a sequence of amino acids characteristic of a specific polypeptide. Alternatively, the mRNA may function as an antisense gene product that inhibits expression of a target gene.

A promoter is a DNA sequence that directs the transcription of a gene, such as a structural gene, an antisense gene, a ribozyme gene or an external guide sequence gene. Typically, a promoter is located in the 5′ region of a gene, proximal to the transcriptional start site. If a promoter is an inducible promoter, then the rate of transcription increases in response to an inducing agent. In contrast, the rate of transcription is not regulated or largely unregulated, by an inducing agent if the promoter is a constitutive promoter. A plant compatible promoter is a promoter sequence that will direct the transcription of a gene in a plant cell.

A core promoter contains essential nucleotide sequences for promoter function, including the TATA box and transcription start site. By this definition, a core promoter may or may not have detectable activity in the absence of specific sequences that may enhance the activity or confer tissue preferred activity. For example, the SGB6 core promoter consists of about 38 nucleotides 5′-ward of the transcriptional start site of the SGB6 gene, while the Cauliflower Mosaic Virus (CaMV) 35S core promoter consists of about 33 nucleotides 5′-ward of the transcriptional start site of the 35S genome.

A tissue-preferred promoter is a DNA sequence that, when operably linked to a gene, directs a higher level of transcription of that gene in a preferred tissue than in some or all other tissues in an organism. For example, an anther-preferred promoter is a DNA sequence that directs a higher level of transcription of an associated gene in plant anther tissue.

An isolated DNA molecule is a fragment of DNA that has been separated from the DNA of an organism. For example, a cloned DNA molecule encoding an avidin gene is an isolated DNA molecule. Another example of an isolated DNA molecule is a chemically-synthesized DNA molecule, or enzymatically-produced cDNA, that is not integrated in the genomic DNA of an organism.

Complementary DNA (cDNA) is a single-stranded DNA molecule that is formed from a mRNA template by the enzyme reverse transcriptase. Typically, a primer complementary to portions of mRNA is employed for the initiation of reverse transcription. Those skilled in the art also use the term “cDNA” to refer to a double-stranded DNA molecule consisting of such a single-stranded DNA molecule and its complementary DNA strand.

The term isoform refers to genetic variants of a polynucleotide which either share the same regulatory function, if the sequence of the polynucleotide spans the regulatory region, or encode protein isoforms with the same function, if the sequence of the polynucleotide covers the coding region. Protein isoforms refer to a set of protein molecules which have the same physical and physiological properties and the same biological function, and whose amino acid sequences have several amino acid differences.

The term operably linked is used to describe the connection between regulatory elements and a gene or its coding region. That is, gene expression is typically placed under the control of certain regulatory elements, including constitutive or inducible promoters, tissue-specific regulatory elements, and enhancers. Such a gene is said to be “operably linked to” or “operatively linked to” the regulatory elements.

The term expression refers to the biosynthesis of a gene product. For example, in the case of a structural gene, expression involves transcription of the structural gene into mRNA and the translation of mRNA into one or more polypeptides.

A cloning vector is a DNA molecule, such as a plasmid, cosmid, or bacteriophage has the capability of replicating autonomously in a host cell. Cloning vectors typically contain one or a small number of restriction endonuclease recognition sites at which foreign DNA sequences can be inserted in a determinable fashion without loss of an essential biological function of the vector, as well as a marker gene that is suitable for use in the identification and selection of cells transformed with the cloning vector. Marker genes typically include genes that provide tetracycline resistance or ampicillin resistance.

An expression vector is a DNA molecule comprising a gene that is expressed in a host cell. Typically, gene expression is placed under the control of certain regulatory elements, including constitutive or inducible promoters, tissue-preferred regulatory elements, and enhancers. Such a gene is said to be “operably linked to” or “operatively linked to” the regulatory elements.

A recombinant host may be any prokaryotic or eukaryotic cell that contains either a cloning vector or expression vector. This term also includes those prokaryotic or eukaryotic cells that have been genetically engineered to contain the cloned gene(s) in the chromosome or genome of the host cell.

A transgenic plant is a plant having one or more plant cells that contain a foreign gene.

In eukaryotes, RNA polymerase II catalyzes the transcription of a structural gene to produce mRNA. A DNA molecule can be designed to contain an RNA polymerase II template in which the RNA transcript has a sequence that is complementary to that of a preferred mRNA. The RNA transcript is termed an antisense RNA and a DNA sequence that encodes the antisense RNA is termed an antisense gene. Antisense RNA molecules inhibit mRNA expression.

Sequence homology is used to describe the sequence relationships between two or more nucleic acids, polynucleotides, or proteins, polypeptides, and is understood in the context and in conjunction with the terms including: (a) “reference sequence,” (b) “comparison window,” (c) “sequence identity,” (d) “percentage of sequence identity,” and (e) “substantial identity” or “homologous.”

(a) A “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) A “comparison window” includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73: 237-244 (1988); Higgins and Sharp, CABIOS 5: 151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997).

As those of ordinary skill in the art will understand, BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Clayerie and States, Comput. Chem., 17:191-201 (1993)) low-complexity filters can be employed alone or in combination.

(c) “Sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

(d) “Percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

(e) (i) The term “substantial identity” or “homologous” means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid.

(e) (ii) The terms “substantial identity” or “homologous” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman and Wunsch, J. Mol. Biol. 48: 443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions which are not identical may differ by conservative amino acid changes.

Hybridizing variants: Nucleic acid variants within the invention also may be described by reference to their physical properties in hybridization. One skilled in the field will recognize that a nucleic acid can be used to identify its complement or homologue, using nucleic acid hybridization techniques. It will also be recognized that hybridization can occur with less than 100% complementarity. However, given appropriate choice of conditions, hybridization techniques can be used to differentiate among DNA sequences based on their structural relatedness to a particular probe. For guidance regarding such conditions see, for example, Sambrook et al., 1989, Molecular Cloning—A Laboratory Manual, 2nd ed., Vol. 1-3; and Ausubel et al., 1989, Current Protocols in Molecular Biology, Green Publishing Associates and Wiley Interscience, N.Y.

Structural relatedness between two polynucleotide sequences can be expressed as a function of “stringency” of the conditions under which the two sequences will hybridize with one another. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences. (Bolton et al., 1962, Proc. Natl. Acad. Sci. 48:1390) Hybridization stringency is thus a function of many factors, including overall DNA concentration, ionic strength, temperature, probe size and the presence of agents that disrupt hydrogen bonding. Factors promoting hybridization include high DNA concentrations, high ionic strengths, low temperatures, longer probe size and the absence of agents that disrupt hydrogen bonding.

Hybridization usually is done in two stages. First, in the “binding” stage, the probe is bound to the target under conditions favoring hybridization. A representative hybridization solution comprises 6×SSC, 0.5% SDS, 5× Denhardt's solution and 100 μg of non-specific carrier DNA. See Ausubel et al., supra, section 2.9, supplement 27 (1994). A stock 20×SSC solution contains 3M sodium chloride, 0.3M sodium citrate, pH 7.0. Of course many different, yet functionally equivalent, buffer conditions are known. For high stringency, the temperature is between about 65° C. and 70° C. in a hybridization solution of 6×SSC, 0.5% SDS, 5× Denhardt's solution and 100 μg of non-specific carrier DNA. Moderate stringency is between at least about 40° C. to less than about 65° C. in the same hybridization solution. In both cases, the preferred probe is 100 bases.

Second, the excess probe is removed by washing, which is most important in determining relatedness via hybridization. Washing solutions typically contain lower salt concentrations. A medium stringency wash solution contains the equivalent in ionic strength of 2×SSC and 0.5-0.1% SDS. A high stringency wash solution contains the equivalent in ionic strength of less than about 0.2×SSC and 0.1% SDS, with a preferred stringent solution containing about 0.1×SSC and 0.1% SDS. The temperatures associated with various stringencies are the same as discussed above for “binding.” The washing solution also typically is replaced a number of times during washing. For example, typical high stringency washing conditions comprise washing with 2×SSC plus 0.05% SDS five times at room temperature, and then washing with 0.1×SSC plus 0.1% SDS at 68° C. for 1 h. Blots containing the hybridized, labeled probe are exposed to film for one to three days.

2. The Pin1 Promoters

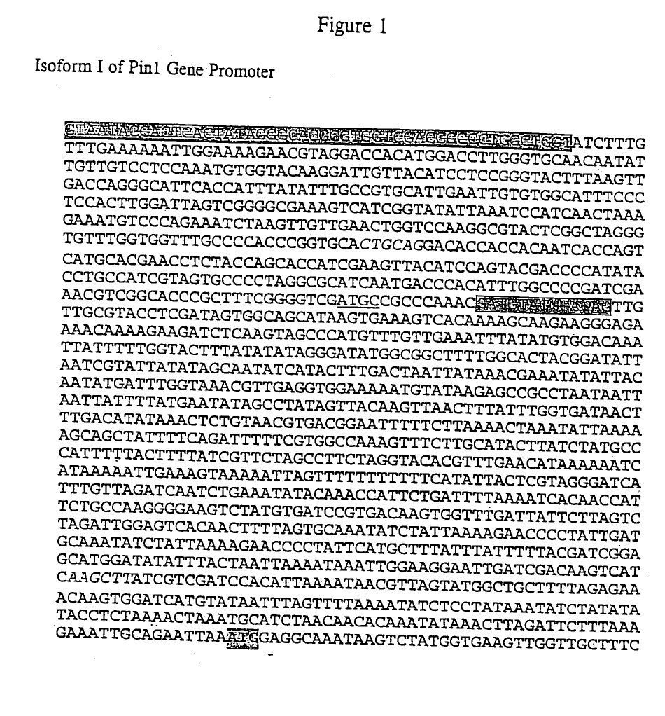

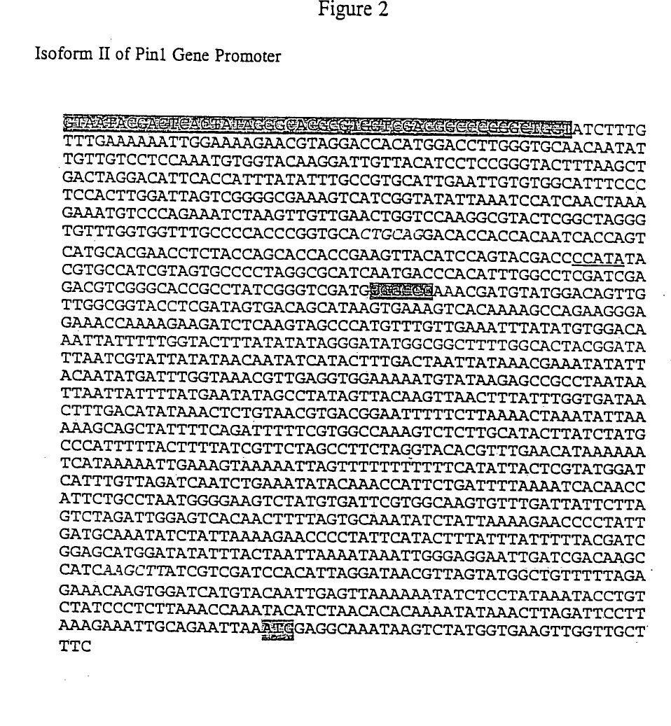

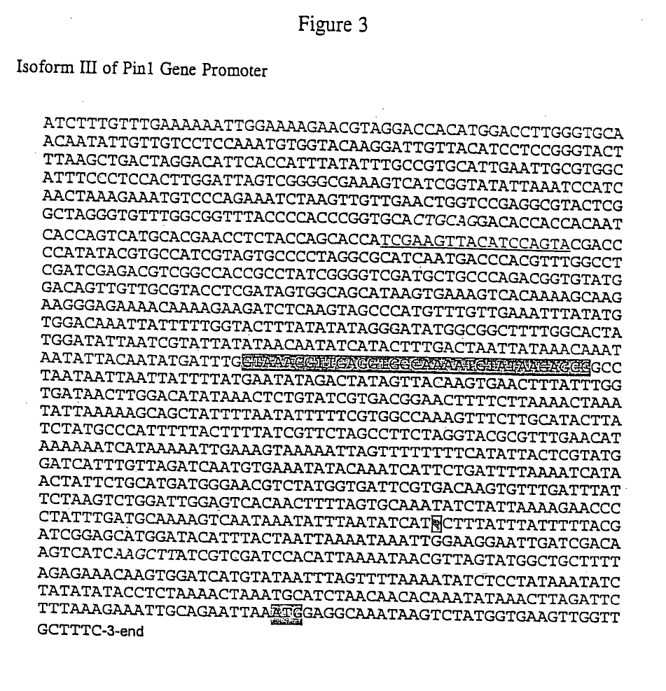

One type of embodied promoter of the present invention is the proteinase inhibitor (pin1) promoter. There are provided four isoforms of the pin1 promoter. The DNA sequence of isoform I, isoform II, and isoform III of the pin1 promoter is disclosed as FIGS. 1, 2, and 3, respectively.

Referring to FIG. 1, for example, the full-length sequence of the pin1 gene promoter isoform I is shown. The shaded area 100 represents the translation start site (ATG). The underlined area 102 represents the TATA box, and the underlined area 104 represents the CAATT box.

The Eukaryotic Promoter Database (http://srs.ebi.ac.uk:9999/srs6bin/cgi-bin/wgetz?-page+LibInfo+-id+4Flds1EqUDE+-lib+EPD) was searched. None of the isoforms of the pin1 gene promoter of this invention exhibits significant sequence homology compared to any eukaryotic promoters reported to date and verified using homology search methods such as BLAST. The highest matching sequence found is gnl|EPD|14007 (+) Pv[dlec2] PHA-L, with an E value of 0.001, which is not significantly homologous to the promoter isoform sequence of this invention.

In one embodiment, the promoter of this invention comprises the entire sequence of isoform I of the pin1 gene promoter, and the various functional segments thereof. In another embodiment, the promoter of this invention comprises the entire sequence of isoform I of the pin1 gene promoter, and the various functional segments thereof. In another embodiment, the promoter of this invention comprises the entire sequence of isoform II of the pin1 gene promoter, and the various functional segments thereof. In yet anther embodiment, the promoter of this invention comprises the entire sequence of isoform III of the pin1 gene promoter, and the various functional segments thereof. In another embodiment, the polynucleotide of the invention has at least 70%, more preferably 80%, most preferably 90%, sequence identity with any one of isoforms I, II, and III and has pin1 gene promoter activity.

The pin1 gene promoter isoforms of the present invention and, any functional segments thereof, may be used in connection with an external enhancer element to achieve high-level of gene expression, as well as to enable the high-level control of the enhanced expression. An enhancer element is cis-acting and is generally upstream from and within 5000 bp of a promoter. The enhancer element is preferably located within about 2000 bp, most preferably adjacent to, or within about 1000 bp of, the transcription initiation codon of the promoter. Conventionally, the initial nucleotide of the transcribed mRNA is designated +1, thus the sequence containing the enhancer is preferably located upstream from about −50 to about −1000 bp, usually from −50 to about −800, and more specifically from −50 to −500 bp from the transcription initiation codon. The enhancer element may be located upstream or downstream in relation to the promoter it affects. Alternatively, the enhancer element may be positioned within introns in a transcription unit.

The external enhancer elements that may be used in conjunction with the promoter elements of the present invention are themselves separately functional. Each individually, in tandem, or dispersed, is independently capable of affecting gene transcription of a promoter operatively linked thereto. In one preferred embodiment, the functional elements of the pin1 gene promoter and the external enhancer elements may be variously combined to provide synergistic effect in increasing the gene transcription capabilities of a promoter operatively linked to these elements. In one further embodiment, the functional elements of the pin1 gene promoter, its isoforms, and the external enhancer elements may be variously combined to confer regulatable control to an operably linked gene.

3. The Amt Promoters

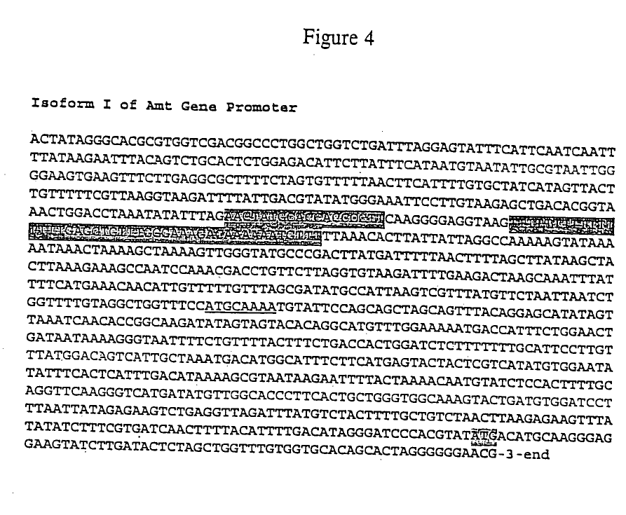

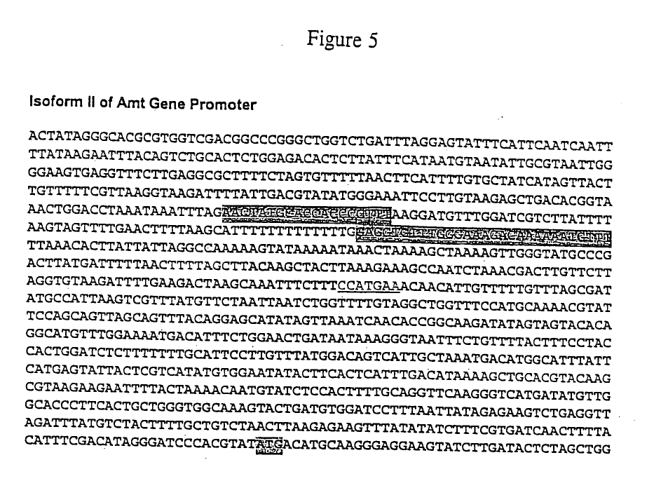

Another type of embodied promoter of the present invention is the aminotransferase (amt) gene promoter. There are provided two isoforms of the amt gene promoter. The full length sequence of isoform I and isoform II of the amt gene promoter is disclosed in FIGS. 4 and 5, respectively.

Referring to FIG. 4, for example, the full-length sequence of the amt gene promoter isoform I is shown. The shaded area 200 represents the translation start site (ATG). The underlined area 202 represents the TATA box, and the underlined area 204 represents the CAATT box.

Neither isoform I nor isoform II of the amt gene promoter of this invention shares any significant sequence homology with any eukaryotic promoters reported to date. Sequence homology searches were performed, for example a BLAST search, using the collection of the promoter sequences in the Eukaryotic Promoter Database (http://srs.ebi.ac.uk:9999/srs6bin/cgi-bin/wgetz?-page+LibInfo+-id+4Flds1EqUDE+-lib+EPD). The highest matching sequence found is gnl|EPD|1005 (+) Am chalcone synthase, with an E value of 0.17, which is not significantly homologous to this promoter isoform sequence.

In one embodiment, the promoter of this invention comprises the entire sequence of isoform I of the amt gene promoter, and the various functional segments thereof. In another embodiment, the promoter of this invention comprises the entire sequence of isoform II of the amt gene promoter, and the various functional segments thereof.

The amt gene promoter isoforms of the present invention, and any functional segments thereof, may be used in connection with an external enhancer element to achieve high-level of gene expression, as well as to enable the high-level control of the enhanced expression.

An enhancer element is cis-acting and may be upstream or downstream of a promoter; it may also be positioned within introns in a transcription unit. The external enhancer elements that may be used in conjunction with the promoter elements of the present invention are themselves separately functional; each individually, in tandem, or dispersed, is independently capable of affecting gene transcription of a promoter operatively linked thereto. In one preferred embodiment, the functional elements of the aminotransferase promoter, i.e., its isoforms I and II, and the external enhancer elements may be variously combined to provide synergistic effect in increasing the gene transcription capabilities of a promoter operatively linked to these elements. In one further embodiment, the functional elements of the aminotransferase promoter—its isoforms—and the external enhancer elements may be variously combined to confer regulatable control to an operably linked gene.

4. Promoter Constructs, Promoter-Enhancer Combinations

In one embodiment, the promoter isoforms of the present invention and their functional elements are operatively linked to an external enhancer. The suitable enhancer may be any plant-compatible enhancer. Operated as such combinations, the overall transcriptional activity may be increased or otherwise modified.

The expression of structural genes employed in the present invention may be operably linked to the promoter-enhancer combinations according to the present invention. The recombinant constructs designed as such may be modified, if desired, to affect their control characteristics.

Environmental factors and hormonal agents may be utilized to test the activities of the promoter constructs according to the present invention, and thus to identify the responsive promoter constructs for various conditions. The transcriptional activities may be determined by measuring the levels of expression of a reporter gene, such as the B-glucoronidase gene (gus), under various conditions. Therefore, the promoter constructs of the present invention may confer regulatory effect upon a structural gene in response to the changes in the exogenous as well as endogenous environment.

The functional elements of the pin1 and amt promoters are identified using methods well known to the skilled artisan. Subclones or oligonucleotides corresponding to fragments of the pin1 or amt promoters are operably linked to screenable markers. The promoter activity of the DNA fragment is tested in transformed cells.

5. Structural Genes

The promoter according to the present invention is operably linked to a gene of interest. The gene usually includes an open reading frame (ORF) encoding a polypeptide or protein having the desired biological activity. Methods for obtaining such genes are well-known to those skilled in the art. For example, open reading frames may be from natural open reading frames encoding protein products, cDNA sequences, synthetic DNA, open reading frames derived from exon ligation, or combinations thereof.

Genes whose level of expression may be increased according to the present invention include, but are not limited to, sequences from the natural genes (plant, animal, bacterial, viral, fungal) which encode primary RNA products; synthetic DNA sequences which encode a specific RNA or protein product; DNA sequences modified by mutagenesis, for example site specific mutagenesis; chimeras of any of the above (to produce fusion proteins); and DNA sequences encoding complementary RNA molecules (antisense), and combinations and/or fragments of the above.

Examples of proteins that can be produced at increased levels utilizing the present invention include, but are not limited to pharmaceuticals; nutritionally important proteins; growth promoting factors; proteins for early flowering in plants; proteins giving protection to the plant under certain environmental conditions, e.g., proteins conferring resistance to metals or other toxic substances, such as herbicides or pesticides; stress related proteins which confer tolerance to temperature extremes; proteins conferring resistance to fungi, bacteria, viruses, insects and nematodes as well as proteins of specific commercial value, e.g., enzymes involved in metabolic pathways, such as EPSP synthase.

6. Method

In one embodiment, the invention provides a method for increasing expression of a gene in a cell. The method comprises operably linking a promoter or functional promoter elements according to the present invention to a gene of interest. In an alternative embodiment, the method comprises operably linking an external enhancer element to a promoter or functional promoter elements of this invention, which is operably linked to a gene of interest. The resulting promoter construct increases the expression of the gene. The terms “increased” or “increasing” as used herein refer to gene expression which is elevated as compared to expression of the corresponding wild type gene that is not associated with a promoter containing an enhancer element according to the present invention.

7. Markers and Vectors

The promoter or promoter elements according to the present invention are especially suitable for the construction of gene expression vectors. Methods for preparing gene expression vectors are well known to those skilled in the art. For example, the expression vector may be a plasmid into which a gene, under the control of a suitable promoter of this invention and other regulatory elements, and encoding a product of interest, has been inserted.

Optionally, a selectable marker may be associated with the construct containing the promoter or promoter elements operatively linked to the structural gene, or alternatively the marker may be associated with the construct containing an enhancer element operatively linked to the promoter or promoter elements of this invention which in turn are operatively linked to the structural gene. As used herein, the term “marker” refers to a gene encoding a trait or a phenotype which permits the selection of, or the screening for, a plant or plant cell containing the marker. Preferably, the marker gene is an antibiotic resistance gene whereby the appropriate antibiotic can be used to select for transformed plant cells from among cells that are not transformed. Examples of suitable selectable markers include adenosine deaminase, dihydrofolate reductase, hygromycin-B-phosphotransferase, thymidine kinase, xanthine-guanine phospho-ribosyltransferase and amino-glycoside 3′-O-phosphotransferase II (kanamycin, neomycin and G418 resistance). Other suitable markers will be known to those of skill in the art. For example, screenable markers, such as the uidA gene, GUS, luciferase or the GFP gene may also be used.

8. Transgenic Plants

Also disclosed are transgenic plants comprising a promoter or promoter elements. The promoter or promoter elements according to the present invention may be used in the same or different species from which it is derived or in which it naturally functions. More preferably, the promoter or promoter elements are used for enhanced gene expression in plants. Most preferably, the promoter or promoter elements according to the present invention are used for non-native gene expression in a plant. By “non-native” gene expression it is meant that the promoter or promoter elements, and the optional enhancer element operatively linked thereto, controls and enables high level expression of a gene that is not normally found in the host plant.

The transformation of plants in accordance with the invention may be carried out in essentially any of the various ways known to those skilled in the art of plant molecular biology. (See, for example, Methods of Enzymology, Vol. 153, 1987, Wu and Grossman, Eds., Academic Press, incorporated herein by reference). As used herein, the term “transformation” refers to alteration of the genotype of a host plant by the introduction of exogenous or endogenous nucleic acid sequences.

To commence a transformation process in accordance with the present invention, it is first necessary to construct a suitable vector and properly introduce the vector into the plant cell. The details of the construction of the vectors utilized herein are known to those skilled in the art of plant genetic engineering.

For example, the promoter constructs utilized in the present invention may be introduced into plant cells using Ti plasmids, root-inducing (Ri) plasmids, and plant virus vectors. For reviews of such techniques see, for example, Weissbach & Weissbach, 1988, Methods for Plant Molecular Biology, Academic Press, N.Y., Section VIII, pp. 421-463; and Grierson & Corey, 1988, Plant Molecular Biology, 2d Ed., Blackie, London, Ch. 7-9, and Florsch et al., Science 227:1229 (1985), both incorporated herein by reference.

A skilled artisan will be able to select an appropriate vector for introducing the nucleic acid sequences of the invention in a relatively intact state. Thus, any vector which will produce a plant carrying the introduced DNA sequence should be sufficient. Even a naked piece of DNA would be expected to be able to confer the properties of this invention, though at low efficiency. The selection of the vector, or whether to use a vector, is typically guided by the method of transformation selected.

For example, a heterologous nucleic acid sequence can be introduced into a plant cell utilizing Agrobacterium tumefaciens containing the Ti plasmid. When using an A. tumefaciens culture as a transformation vehicle, it is most advantageous to use a non-oncogenic strain of the Agrobacterium as the vector carrier so that normal non-oncogenic differentiation of the transformed tissues is possible. It is also preferred that the Agrobacterium harbor a binary Ti plasmid system. Such a binary system comprises 1) a first Ti plasmid having a virulence region essential for the introduction of transfer DNA (T-DNA) into plants, and 2) a chimeric plasmid. The chimeric plasmid contains at least one border region of the T-DNA region of a wild-type Ti plasmid flanking the nucleic acid to be transferred. Binary Ti plasmid systems have been shown effective to transform plant cells (De Framond, Biotechnology, 1:262, 1983; Hoekema et al., Nature 303:179 (1983). Such a binary system is preferred because it does not require integration into Ti plasmid in Agrobacterium.

Methods involving the use of Agrobacterium include, but are not limited to: 1) co-cultivation of Agrobacterium with cultured isolated protoplasts; 2) transformation of plant cells or tissues with Agrobacterium; or 3) transformation of seeds, apices or meristems with Agrobacterium.

In addition, gene transfer can be accomplished by in situ transformation by Agrobacterium, as described by Bechtold et al., C. R. Acad. Sci. Paris 316:1194 (1993). This approach is based on the vacuum infiltration of a suspension of Agrobacterium cells.

Alternatively, the promoter construct according to this invention can be introduced into a plant cell by contacting the plant cell using mechanical or chemical means. For example, nucleic acid can be mechanically transferred by direct microinjection into plant cells utilizing micropipettes. Moreover, the nucleic acid may be transferred into plant cells using polyethylene glycol which forms a precipitation complex with genetic material that is taken up by the cell.

The nucleic acid can also be introduced into plant cells by electroporation (Fromm et al., Proc. Natl. Acad. Sci., U.S.A. 82:5824 (1985), which is incorporated herein by reference). In this technique, plant protoplasts are electroporated in the presence of vectors or nucleic acids containing the relevant nucleic acid sequences. Electrical impulses of high field strength reversibly permeabilize plant membranes allowing the introduction of nucleic acids. Electroporated plant protoplasts reform the cell wall, divide and form a plant callus. Selection of the transformed plant cells with the transformed gene can be accomplished using phenotypic markers as described herein.

Another method for introducing nucleic acid into a plant cell is high velocity microprojectile bombardment with small particles with the nucleic acid to be introduced contained either within the matrix of small beads or particles, or on the surface thereof (Klein et al., Nature 327:70 (1987). Although, typically only a single introduction of a new nucleic acid sequence is required, this method particularly provides for multiple introductions.

Cauliflower mosaic virus (CaMV) may also be used as a vector for introducing heterologous nucleic acid into plant cells (U.S. Pat. No. 4,407,956). The CaMV viral DNA genome is inserted into a parent bacterial plasmid creating a recombinant DNA molecule which can be propagated in bacteria. After cloning, the recombinant plasmid may be re-cloned and further modified by introduction of the desired nucleic acid sequence. The modified viral portion of the recombinant plasmid is then excised from the parent bacterial plasmid, and used to inoculate the plant cells or plants.

9. Production of Proteins Using Transgenic Plants

The vectors of this invention may be used to facilitate the expression and/or secretion of heterologous proteins in cell culture or by crop cultivation.

Plant cells comprising an expression vector for high level expression of the protein product of interest, are placed and maintained in suspension culture, and induced through the variety of inducers, suitable for the promoters used to construct the expression vectors described above, to produce high levels of the desired heterologous protein. The protein is then isolated using conventional technology.

Alternatively, plant cells comprising the expression vector for high level expression of the protein of interest, may be regenerated into transgenic plants as described above. Suitable plant parts of the plant are then harvested and the protein product isolated using conventional technology.

Because the purification steps differ from protein to protein, it is sufficient to indicate that the initial purification process typically will be similar to the purification process for the native protein from its host. Because the growth media of the plant suspension culture, as used in the present invention, is typically more simple than the normal host environment of the protein of interest, the purification procedures may be appropriately modified and simplified by those of skill in the art.

By combining the technology of the present invention with well-established production methods (e.g., plant cell fermentation, crop cultivation, and product recovery), recombinant protein can be efficiently and economically produced for the biopharmaceutical, industrial processing, animal health and bioremediation industries.

10. Proteinase Inhibitor 1 and Aminotransferase Activity

Proteinase inhibitors are a family of proteins whose function is to prevent unwanted proteolysis in the tissues of both animals and plants. In plants, proteinase inhibitors generally reduce the nutritional quality of plant organs and their presence is thought to represent a defense against herbivorous insects. These proteins typically accumulate in tissues where wounding has occurred. The potato proteinase inhibitor I gene exhibits significant homology to the ethylene responsive proteinase inhibitor I gene in tomato. This group of proteins accumulate preferentially in ripening fruit, rather than wounded fruit Margossian et al., 85:8012 (1988). Proteinase inhibitors may be used in general to prevent unwanted proteolysis and stabilize protein-bearing solutions. These specific proteins may be useful in ex vivo biological processing (e.g., fermentation, purifications, stabilization for storage, among others). In addition, proteinase inhibitors may be used in transgenic plants to afford protection against proteolysis (e.g., protection of plant portions, enhancements for plant-based recombinant protein production, etc.).

The presence of proteinase inhibitors may be determined immunologically via Western blot assay. Pearce et al., Planta 175:527 (1988). In addition, proteinase inhibitor activity has been measured against aggressive proteases such as trypsin and chymotrypsin with substrates tosyl-L-arginine methyl ester or benzoyl-L-tyrosine ethyl ester. Pearce et (1988) supra; Hummel, Can J Biochem 37:1393 (1959).

Aminotransferases catalyze transamination reactions in planta involving important amino acids including glutamate, aspartate, alanine, valine, leucine and isoleucine Wightman, Phytochem. 17:1455 (1978). Plant aminotransferases have been shown be active in vitro and may have direct impact in bioprocessing applications as well as in combinatorial biosynthesis both in vitro and in planta.

An aminotransferase enzyme fraction can be extracted from plant tissues by homogenization of plant tissues and suspension in Tris-HCl buffer as reported by Forest et al., Can J Biochem 50:538 (1972). In this report, enzyme buffer fraction from bushbean (Phaseolus vulgaris) contained virtually all of the aminotransferase activity, with no detectable enzyme in the solid phase. Further, transaminase activity was measured by mixing one of 22 amino acids as the amino group donor with a-ketoglutarate, oxaloacetate, pyruvate or glyoxylate as the amino group and subsequently determining reaction product. Forest et al. (1972) supra; Wilson et al., J Biol Chem 208:863 (1954). Composition of the new reaction mixture may be determined chromatographically or using colorimetric methods. Forest et al., Can J Biochem 49:709 (1971).

11. Application in Controlled Environment Agriculture

In one embodiment of this invention, the promoter and promoter constructs of the present invention may be used in CEA. CEA employs an integrated system for commercial production of a heterologous protein in transgenic plants in controlled environment. Plants are grown under defined environmental conditions, for example in a greenhouse, to optimize growth of the transgenic plant as well as expression of the gene encoding the heterologous protein. In CEA, the transgenic plants may be cultivated through hydroponics in soil-less or soil-containing media. The transgenic plants selected for heterologous protein production under the defined environmental conditions of CEA may also be grown in open field agriculture (OFA) to produce the protein of interest. Diverse plant species may be used including dicots and monocots.

The transgenic plants used in CEA according to the present invention are transformed with an expression vector comprising a CEA promoter operably linked to a gene encoding the heterologous protein of interest. The aforementioned promoter or promoter elements of this invention are used as the CEA promoter in this context; the selected CEA promoter maximizes heterologous protein production under the corresponding environmental condition of CEA.

The following Examples 3 and 4 demonstrate that, under controlled and modified conditions, expression of relevant genes is enhanced. Such enhancement often can correlate with increased accumulation of the encoded proteins. Example 5 further discusses the use of the promoters of this invention in increasing or otherwise controlling protein production in CEA.

### EXAMPLE 1

### Senescence-Active cDNA Library Construction

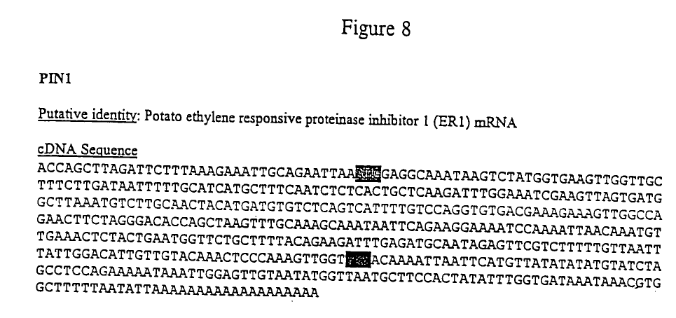

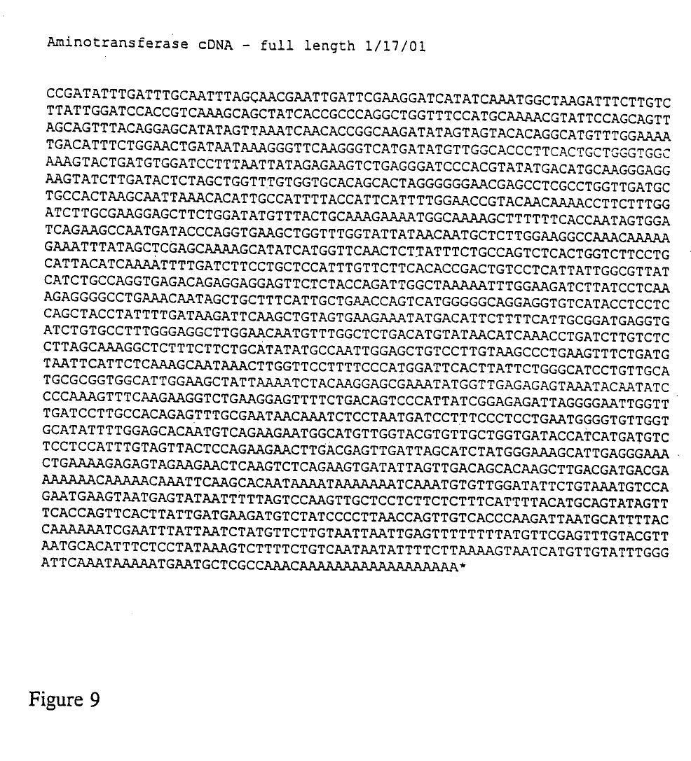

A cDNA library was constructed from poly(A) RNA isolated from dark-treated excised potato leaf tissues using the ZAP cDNA synthesis kit (Strategene). Approximately 60,000 plaques from the cDNA library were plated, transferred onto duplicate nitrocellulose filters, hibridized with radiolabelled cDNA probes synthesized independently from 1 μg poly (A) RNA of healthy untreated and dark-treated leaf tissues. Plaques showing contrasting signal intensity between untreated and dark-treated probing were collected, re-plated, and rescreened using newly synthesized cDNA probes. A single, pure plaque from each of these clones still demonstrating a differential hybridization signal intensity was collected, and the pBluescript phagemid containing the cDNA insert was excised from the mUniZAP vector as described by the manufacturer (Stratagene). Nucleotide sequences were determined by automated sequencing. Homology-based searches of the Genebank databases were performed using the BLASTN program. Altshul et al., J. Mol. Biol. 215: 403 (1990). The DNA sequence of the cDNA corresponding to the pin1 gene is shown in FIG. 8. The DNA sequence of the cDNA corresponding to the amt gene is shown in FIG. 9.

### EXAMPLE 2

### Isolation of the Pin1 and Amt Gene Promoters

The promoter elements of pin1 and amt were isolated using the Genome Walker™ kit (CLONETECH). Briefly, potato genomic DNA was first digested with restriction endonucleases: Dra I, Eco R V, Pvu II, Sca 1, and SspI. An adapter was ligated onto the digested genomic DNA fragments to create five libraries of potato specific genomic DNA fragments corresponding to the restriction endonucleases. The genomic libraries were then used as templates in nested PCR reactions with gene-specific primers (pin1 or amt) and the adaptor primers provided from manufacturer. The PCR products were cloned into pGEM vectors for DNA fragment amplification and sequencing. The promoter elements were confirmed by comparison with known cDNA sequences since the gene-specific primers were designed about 100 to 100 bp downstream of the cDNA clone.

Primers used for promoter isolation are as follows:

Genome Walker Adapter primers from CLONETECH

| | |
|---|---|
| Adapter primer 1 (AP1): | 5′-GTA ATA CGA CTC ACT ATA GGG C-3′ |
| Nested adapter primer 2 (AP2): | 5′-ACT ATA GGG CAC GCG TGG T-3′ |

For the proteinase inhibitor gene (pin1)

| | |
|---|---|
| Primer SEN16 (77-51 antisense) | 5-GAA AGC AAC CAA CTT CAC CAT AGA CT-3′ |
| Primer SEN28 (65-28 antisense): | 5′-CTT CAC CAT AGA CTT ATT TGC CTC |
| | CAT TTA ATT CTG CA-3′ |

For the aminotransferase gene (amt)

| | |
|---|---|
| Primer SEN29 (119-93): | 5′-CCA GCT AGA GTA TCA AGA TAC TTC CT-3′ |
| Primer SEN30 (148-120): | 5′-CGT TCC CCC CTA GTG CTG TGC ACC ACA A-3′ |
| Primer SEN31 (178-148): | 5′-GCT TAG TGG CAG CAT CAA CCA GGC GAG GCT-3′ |

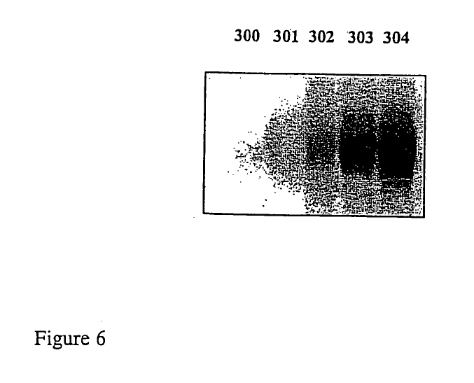

The DNA sequence of the pin1 and amt genomic clones was determined by automatic sequencing of the DNA Sequencing Facility, Iowa State University, Ames, Iowa. The DNA sequences of isoforms I, II, and III of the pin1 gene promoter are shown in FIGS. 1, 2, and 3, respectively. The DNA sequences of isoform I and II of the amt gene promoter are shown in FIGS. 5 and 6, respectively.

### EXAMPLE 3

### Northern Analysis of Amt Gene Expression in Potato Incubated in the Dark

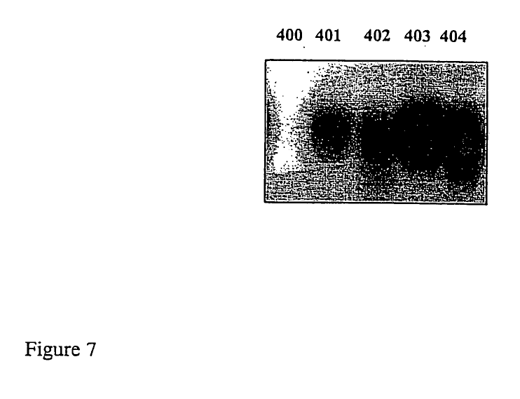

The amt gene promoter of this invention confers light/dark sensitivity to the amt gene as reflected in the Northern analysis. Referring to FIG. 7, dark induces senescence and the enhanced expression of amt gene driven by the promoter disclosed herein. The bands 301, 302, 303, and 304 represent the levels of expressed gene product after dark treatment of 1 day, 2 days, 3 days, and 4 days, respectively. The first lane (300) is the control, the sample for which was taken before the dark treatment. The potato leaves were cut and maintained in the 10 mM MES buffer and treated with 100 ppm ethylene at room temperature and kept in dark. A 30 ug aliquot of total RNA extracted from the treated leaves was used for electrophoresis on 1.3% of formaldehyde agarose gels. The target mRNA was fixed on the Zeta probe membrane at 65° C. for 17 hrs. A ³²P labeled probe, a Hind III cDNA fragment of potato aminotransferase gene, was used to hybridize the membrane. After washing, x-ray film was exposed to the membranes for 2 days, and the resulting exposure for each band was measured. As revealed by Northern analysis, the pin1 gene promoter is induced by dark treatment.

### EXAMPLE 4

### Northern Analysis of Pin1 Gene Expression in Potato Incubated in the Dark

FIG. 6 demonstrates an example of Northern analysis for expression of the pin1 gene controlled by the native pin1 gene promoter. Plants were exposed to the dark for 0, 1, 2, 3, and 4 days and gene expression monitored by Northern analysis with the results shown in lanes 400, 401, 402, 403, and 404, respectively. The experimental protocol was similar to that described in Example 3, except that an EcoR I/Xho I restriction fragment of pin1 gene was used as the probe. As revealed by Northern analysis, the pin1 gene promoter is induced by dark treatment.

### EXAMPLE 5

### Increased or Controlled Protein Production in CEA Using the Pin1 Gene Promoter

Essential to enable protein production in CEA, the responsiveness to environmentally controllable conditions may be conferred by the promoter or promoter elements of this invention to a target gene. The target gene may be the native gene, or it may encode another protein product of interest.

This example describes gene cloning and stable transformation assays of the expression of GUS reporter gene under the control of the pin1 gene promoter. Substituting GUS with a gene encoding a functional protein of interest, this system may be used in CEA to increase or control the production of the functional protein of interest. This system can also be used in general to study the function of promoter fragments by evaluating the transcriptional activity of a reporter gene operably linked to those fragments.

Bacterium Strain and Plant Materials

Escherichia coli DH5α was used as the host for routine cloning experiments. The A. tumefaciens strain PC2760 was the host for the binary vectors. N. tabacum cell suspension culture designated NT1 was used for cell culture experiments. Solanum tuberosum cv. Desiree was used as the host for Agrobacterium-mediated transformation and for genomic DNA isolation.

Construction of PIN1 Promoter:Gus Fusion Genes

To prepare the transcriptional fusion construct pin1:gus, the 1.5 kb pin1 promoter fragment was amplified by PCR using pin1 as a DNA template and placed one nucleotide upstream of the putative translation start site. The pin1 promoter/gus reporter gene fragment was cloned as a cassette into the plant/cell culture expression vector pGA482 (An et al. 1987. Meth. Enzymol. 153:293-305), producing pin1:gus for stable transformation in tobacco cell culture and potato plants.

Fluorometric Analysis of GUS Activity

Fluorometric quantitation of GUS activity was performed according to Jefferson et al., EMBO J. 6:3901-3907 (1987). NT1 cell culture protein was extracted in lysis buffer (50 mM sodium phosphate, pH 7.0, 10 mM EDTA, 0.1% TritonX-100, 0.1% sarkosyl and 10 mM DTT) by 2× sonication on ice for 5 seconds. Potato plant protein was extracted by grinding in the same buffer. Protein concentrations in cell culture and plant extracts were determined by the Bio-Rad method, Bradford, Anal. Biochem. 72:248-254 (1976). Approximately 5-10 μg of protein was incubated in the presence of 1 mM 4-methylumbelliferyl β-D-glucuronide in 100 μl of lysis buffer at 37C. Samples were taken at 0, 15, and 30 min and the enzymatic reaction was quenched in 0.2 M sodium carbonate (Na₂CO₃). The fluorometer was calibrated with 100, 200, 300, and 400 nM 4-methylumbelliferon in 0.2M sodium carbonate.

Stable Transformation of Pin1Gus

The transcriptional fusion construct pin1:gus was prepared by inserting the pin1 promoter:gus reporter gene fragment as a cassette into a plant expression binary vector. The chimeric promoter/gus construct was transferred into tobacco cell culture and potato plants via Agrobacterium transformation. Gus expression in at least 100 independently transformed calli and 30 independently transformed plants were regenerated. Gus levels in transgenic calli were measured in normal growth, dark-treatment and ethylene-treatment conditions. In addition, Gus levels in transgenic primary transformants were measured under normal growth, dark treatment, ethylene treatment, −abscission, abscission+dark treatment and abscission+ethylene treatment conditions.

The protein production system of CEA therefore comprises a transgenic plant transformed with an expression vector comprising a CEA promoter operably linked to a gene encoding the heterologous protein of interest. Preferably, the plant used in this protein production system is selected because under conditions of CEA it produces (1) rapid and efficient growth of harvested plant biomass containing the heterologous protein; (2) large amounts of heterologous protein in the harvested plant biomass; and (3) a plant tissue extract wherein the heterologous protein is stable.

### EXAMPLE 6

### Full Length cDNA of Pin1 Gene

Referring to FIG. 8, the full length cDNA sequence of pin1 gene is illustrated. Sequence homology searches were performed using the nucleic acid sequence database GenBank. It was found that this sequence is highly homologous to the sequence of tomato fruit-ripening protein, i.e., ethylene responsive proteinase inhibitor I (er1) mRNA, with an E value of 0. This result was further confirmed by homology searches using the amino acid sequence database Swisprot. There, tomato ethylene-responsive proteinase inhibitor 1 precursor was identified as homologous with an extremely low E value (4e-59). The identity of the cDNA as encoding potato ethylene responsive proteinase inhibitor is therefore established. Northern analysis further verifies this result.

### EXAMPLE 7

### Northern Analysis of Pin1 Gene Expression with Ethylene Treatment

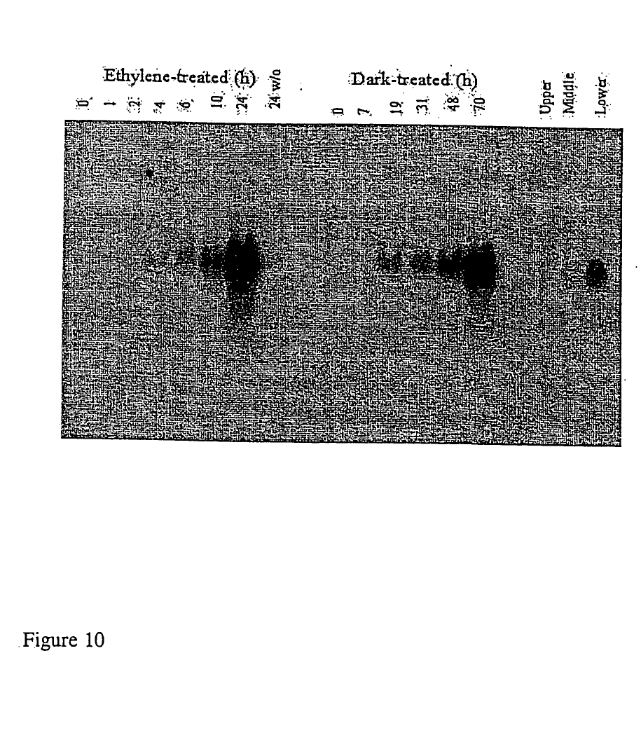

FIG. 10 demonstrates an example of Northern analysis on the expression of pin1 gene controlled by the native pin1 promoter. Ethylene treatment for 1-24 hours at a concentration of 50 ppm was exerted and the accumulation of transcripts was measured at the time points of 1 hour (Lane 702), 2 hours (Lane 704), 4 hours (Lane 706), 6 hours (Lane 708), 10 hours (Lane 710), and 24 hours (Lane 712). Lane 700 represents the zero time point; and Lane 714 represents the control where no ethylene was applied. Increased levels of expression of the gene product are observed after 4-hour treatment, in lanes 706, 708, 710, and 712, respectively. The Northern analysis was similar to that in Example 3 except that an EcoR I/Xho I restriction fragment of pin1 gene was used as the probe.

### EXAMPLE 8

### Full Length cDNA of Amt Gene

Aminotransferases catalyze the transfer of an amino group, plus a proton and an electron pair, from an amino donor compound to the carbonyl position of an amino acceptor compound. Most transaminations are freely reversible but cases of unidirectional (irreversible) transamination are known (Givan, 1980). Aminotransferases are divided into four subgroups on the basis of their mutual structural relatedness (Mehta et al., 1993). Subgroup I comprises aspartate, alanine, tyrosine, histidinol-phosphate, and phenylalanine aminotransferases; subgroup II acetylornithine, ornithine, omega-amino acid, 4-aminobutyrate and diaminopelargonate aminotransferases; subgroup III D-alanine and branched-chain amino acid aminotransferases, and subgroup IV serine and phosphoserine aminotransferases (Mehta et al., 1993). One of the best characterized plant aminotransferase is glutamate-oxaloacetate transaminase (GOT) (or aspartate aminotransferase (AAT) [EC 2.6.1.1]) which catalyzes the reversible interconversions of glutamate and aspartate, and their 2-keto analogs: Glutamate+oxaloacetate (OAA)<--->2-oxoglutarate+aspartate. A cDNA encoding the cytosolic form of this enzyme has been cloned from carrot (Turano et al., 1992). The enzyme is a dimer with 2 identical subunits (40 to 45 kDa).

The present inventors identified the full length cDNA sequence of a amt gene as shown in FIG. 9. Sequence homology searches were performed using the nucleic acid sequence database GenBank. It was shown that this sequence is highly homologous to the sequence of capsicum Chinese strain habanero putative aminotransferase mRNA, with an E value of 0. This result was further confirmed by homology searches using the amino acid sequence database Swisprot. The aminotransferase-like protein from Arabidopsis thaliana was identified as homologous with an E value of 0. The identity of the cDNA as encoding potato aminotransferase is therefore established. Northern analysis was also performed with this gene.

### EXAMPLE 9

### Northern Analysis of the Amt Gene Expression with Ethylene Treatment

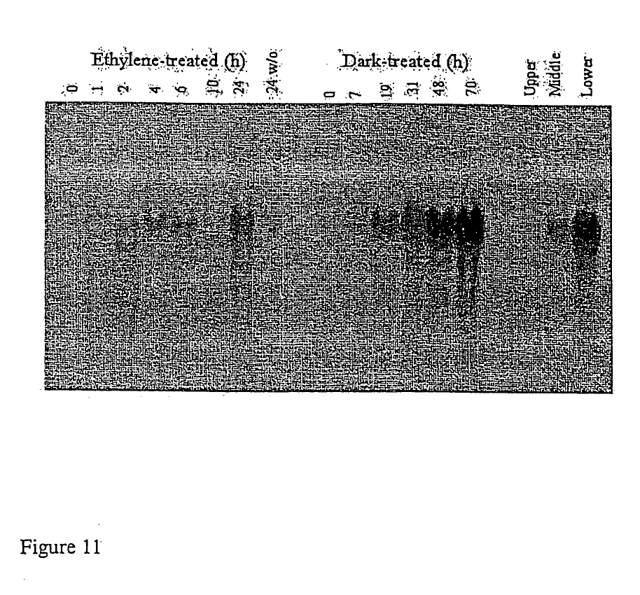

FIG. 11 is an example of Northern analysis on the expression of the amt gene controlled by the native amt gene promoter. Ethylene treatment for 1-24 hours at a concentration of 50 ppm was exerted and the accumulation of transcripts was measured at the time points of 1 hour (Lane 802), 2 hours (Lane 804), 4 hours (Lane 806), 6 hours (Lane 808), 10 hours (Lane 810), and 24 hours (Lane 812). Lane 800 represents the zero time point; and Lane 814 represents the control where no ethylene was applied. Increased levels of expression of the gene product are observed in lanes 804, 806, 808, and 812, for example. The apparent low level of signal in Lane 810 at 10 hours may be due to insufficient sample load. The experimental protocol was similar to that in Example 3, except that a HindIII restriction fragment of potato amt gene was used as the probe.

1-2. (cancelled)

3. An isolated polynucleotide having at least 70% sequence identity with SEQ ID NO.:4 and amt gene promoter activity.

4. An isolated DNA sequence comprising a polynucleotide molecule selected from the group consisting of SEQ ID NO.:4, SEQ ID NO.:5 and functional fragments thereof having amt gene promoter activity.

5. (Cancelled)

6. An expression vector comprising the polynucleotide according to claim 3.

7. (Cancelled)

8. A plant cell comprising the expression vector of claim 6.

9. (Cancelled)

10. A transgenic plant comprising the plant cell of claim 8.

11-12. (Cancelled).

13. A method for producing a gene product in a transformed plant cell comprising the steps of:
(a) constructing a chimeric gene comprising a polynucleotide having at least 70% sequence identity with SEQ ID NO.:4 and amt gene promoter activity operably linked to a structural gene;
(b) transforming a plant cell with the chimeric gene; and
(c) expressing the chimeric gene in the transformed plant cell to produce the gene product.

14. The method according to claim 13, wherein the nucleotide sequence having amt gene promoter activity is selected from the group consisting of SEQ ID NO.:4; SEQ ID NO.:5 that shown in FIG. 4, 6 and any functional fragments thereof having amt gene promoter activity.

15-17. (Cancelled)


SEQUENCE LISTING (14 sequences)

SEQ ID NO: 1 - DNA, 1595 bp, Solanum tuberosum
gtaatacgactcactatagggcacgcgtggtcgacggccctggctggtatctttgtttgaaaaaattggaaaagaacgtaggaccacatggaccttgggtgcaacaatattgttgtcctccaaatgtggtacaaggattgttacatcctccgggtactttaagttgaccagggcattcaccatttatatttgccgtgcattgaattgtgtggcatttccctccacttggattagtcggggcgaaagtcatcggtatattaaatccatcaactaaagaaatgtcccagaaatctaagttgttgaactggtccaaggcgtactcggctagggtgtttggtggtttgccccacccggtgcactgcaggacaccaccacaatcaccagtcatgcacgaacctctaccagcaccatcgaagttacatccagtacgaccccatatacctgccatcgtagtgcccctaggcgcatcaatgacccacatttggccccgatcgaaacgtcggcacccgctttcggggtcgatgccgcccaaacgatgtatggacagttgttgcgtacctcgatagtggcagcataagtgaaagtcacaaaagcaagaagggagaaaacaaaagaagatctcaagtagcccatgtttgttgaaatttatatgtggacaaattatttttggtactttatatatagggatatggcggcttttggcactacggatattaatcgtattatatagcaatatcatactttgactaattataaacgaaatatattacaatatgatttggtaaacgttgaggtggaaaaatgtataagagccgcctaataattaattattttatgaatatagcctatagttacaagttaactttatttggtgataactttgacatataaactctgtaacgtgacggaatttttcttaaaactaaatattaaaaagcagctattttcagatttttcgtggccaaagtttcttgcatacttatctatgcccatttttacttttatcgttctagccttctaggtacacgtttgaacataaaaaatcataaaaattgaaagtaaaaattagtttttttttttcatattactcgtagggatcatttgttagatcaatctgaaatatacaaaccattctgattttaaaatcacaaccattctgccaaggggaagtctatgtgatccgtgacaagtggtttgattattcttagtctagattggagtcacaacttttagtgcaaatatctattaaaagaacccctattgatgcaaatatctattaaaagaacccctattcatgctttatttatttttacgatcggagcatggatatatttactaattaaaataaattggaaggaattgatcgacaagtcatcaagcttatcgtcgatccacattaaaataacgttagtatggctgcttttagagaaacaagtggatcatgtataatttagttttaaaatatctcctataaatatctatatatacctctaaaactaaatgcatctaacaacacaaatataaacttagattctttaaagaaattgcagaattaaatggaggcaaataagtctatggtgaagttggttgctttc

SEQ ID NO: 2 - DNA, 1598 bp, Solanum tuberosum
gtaatacgactcactatagggcacgcgtggtcgacggcccgggctggtatctttgtttgaaaaaattggaaaagaacgtaggaccacatggaccttgggtgcaacaatattgttgtcctccaaatgtggtacaaggattgttacatcctccgggtactttaagctgactaggacattcaccatttatatttgccgtgcattgaattgtgtggcatttccctccacttggattagtcggggcgaaagtcatcggtatattaaatccatcaactaaagaaatgtcccagaaatctaagttgttgaactggtccaaggcgtactcggctagggtgtttggtggtttgccccacccggtgcactgcaggacaccaccacaatcaccagtcatgcacgaacctctaccagcaccaccgaagttacatccagtacgaccccatatacgtgccatcgtagtgcccctaggcgcatcaatgacccacatttggcctcgatcgagacgtcgggcaccgcctatcgggtcgatgccgcccaaacgatgtatggacagttgttggcggtacctcgatagtgacagcataagtgaaagtcacaaaagccagaagggagaaaccaaaagaagatctcaagtagcccatgtttgttgaaatttatatgtggacaaattatttttggtactttatatatagggatatggcggcttttggcactacggatattaatcgtattatataacaatatcatactttgactaattataaacgaaatatattacaatatgatttggtaaacgttgaggtggaaaaatgtataagagccgcctaataattaattattttatgaatatagcctatagttacaagttaactttatttggtgataactttgacatataaactctgtaacgtgacggaatttttcttaaaactaaatattaaaaagcagctattttcacatttttcgtggccaaagtctcttgcatacttatctatgcccatttttacttttatcgttctagccttctaggtacacgtttgaacataaaaaatcataaaaattgaaagtaaaaattagtttttttttttcatattactcgtatggatcatttgttagatcaatctgaaatatacaaaccattctgattttaaaatcacaaccattctgcctaatggggaagtctatgtgattcgtggcaagtgtttgattattcttagtctagattggagtcacaacttttagtgcaaatatctattaaaagaacccctattgatgcaaatatctattaaaagaacccctattcatactttatttatttttacgatcggagcatggatatatttactaattaaaataaattgggaggaattgatcgacaagccatcaagcttatcgtcgatccacattaggataacgttagtatggctgtttttagagaaacaagtggatcatgtacaattgagttaaaaaatatctcctataaatacctgtctatccctcttaaaccaaatacatctaacacacaaaatataaacttagattccttaaagaaattgcagaattaaatggaggcaaataagtctatggtgaagttggttgctttc

SEQ ID NO: 3 - DNA, 1546 bp, Solanum tuberosum
  modified_base (1248): a, t, c or g
atctttgtttgaaaaaattggaaaagaacgtaggaccacatggaccttgggtgcaacaatattgttgtcctccaaatgtggtacaaggattgttacatcctccgggtactttaagctgactaggacattcaccatttatatttgccgtgcattgaattgcgtggcatttccctccacttggattagtcggggcgaaagtcatcggtatattaaatccatcaactaaagaaatgtcccagaaatctaagttgttgaactggtccgaggcgtactcggctagggtgtttggcggtttaccccacccggtgcactgcaggacaccaccacaatcaccagtcatgcacgaacctctaccagcaccatcgaagttacatccagtacgaccccatatacgtgccatcgtagtgcccctaggcgcatcaatgacccacgtttggcctcgatcgagacgtcggccaccgcctatcggggtcgatgctgcccagacggtgtatggacagttgttgcgtacctcgatagtggcagcataagtgaaagtcacaaaagcaagaagggagaaaacaaaagaagatctcaagtagcccatgtttgttgaaatttatatgtggacaaattatttttggtactttatatatagggatatggcggcttttggcactatggatattaatcgtattatataacaatatcatactttgactaattataaacaaataatattacaatatgatttggtaaacgttgaggtggcaaaatgtataagagccgcctaataattaattattttatgaatatagactatagttacaagtgaactttatttggtgataacttggacatataaactctgtatcgtgacggaacttttcttaaaactaaatattaaaaagcagctattttaatatttttcgtggccaaagtttcttgcatacttatctatgcccatttttacttttatcgttctagccttctaggtacgcgtttgaacataaaaaatcataaaaattgaaagtaaaaattagttttttttcatattactcgtatggatcatttgttagatcaatgtgaaatatacaaatcattctgattttaaaatcataactattctgcatgatgggaacgtctatggtgattcgtgacaagtgtttgatttattctaagtctggattggagtcacaacttttagtgcaaatatctattaaaagaacccctatttgatgcaaaagtcaataaatatttaatatcatnctttatttatttttacgatcggagcatggatacatttactaattaaaataaattggaaggaattgatcgacaagtcatcaagcttatcgtcgatccacattcccctaacgttagtatggctgcttttagagaaacaagtggatcatgtataatttagttttcccctatctcctataaatatctatatatacctctaaaactaaatgcatctaacaacacaaatataaacttagattctttaaagaaattgcagaattaaatggaggcaaataagtctatggtgaagttggttgctttc

SEQ ID NO: 4 - DNA, 1175 bp, Solanum tuberosum
actatagggcacgcgtggtcgacggccctggctggtctgatttaggagtatttcattcaatcaattttataagaatttacagtctgcactctggagacattcttatttcataatgtaatattgcgtaattggggaagtgaagtttcttgaggcgcttttctagtgtttttaacttcattttgtgctatcatagttacttgtttttcgttaaggtaagattttattgacgtatatgggaaattccttgtaagagctgacacggtaaactggacctaaatatatttagaactatgcaccaccccttcaaggggaggtaagttttttttttttttttgaggtgtttgggaaagacaaaaaatgtttttaaacacttattattaggccaaaaagtataaaaataaactaaaagctaaaagttgggtatgcccgacttatgatttttaacttttagcttataagctacttaaagaaagccaatccaaacgacctgttcttaggtgtaagattttgaagactaagcaaatttattttcatgaaacaacattgtttttgtttagcgatatgccattaagtcgtttatgttctaattaatctggttttgtaggctggtttccatgcaaaatgtattccagcagctagcagtttacaggagcatatagttaaatcaacaccggcaagatatagtagtacacaggcatgtttggaaaaatgaccatttctggaactgataataaaagggtaattttctgttttactttctgaccactggatctctttttttgcattccttgtttatggacagtcattgctaaatgacatggcatttcttcatgagtactactcgtcatatgtggaatatatttcactcatttgacataaaagcgtaataagaattttactaaaacaatgtatctccacttttgcaggttcaagggtcatgatatgttggcacccttcactgctgggtggcaaagtactgatgtggatcctttaattatagagaagtctgaggttagatttatgtctacttttgctgtctaacttaagagaagtttatatatctttcgtgatcaacttttacattttgacatagggatcccacgtatatgacatgcaagggaggaagtatcttgatactctagctggtttgtggtgcacagcactaggggggaacg

SEQ ID NO: 5 - DNA, 1188 bp, Solanum tuberosum
actatagggcacgcgtggtcgacggcccgggctggtctgatttaggagtatttcattcaatcaattttataagaatttacagtctgcactctggagacactcttatttcataatgtaatattgcgtaattggggaagtgaggtttcttgaggcgcttttctagtgtttttaacttcattttgtgctatcatagttacttgtttttcgttaaggtaagattttattgacgtatatgggaaattccttgtaagagctgacacggtaaactggacctaaataaatttagaactatgcaccacccctttaaggatgtttggatcgtcttattttaagtagttttgaacttttaagcatttttttttttttggaggtgtttgggaaagacaaaaaatgtttttaaacacttattattaggccaaaaagtataaaaataaactaaaagctaaaagttgggtatgcccgacttatgatttttaacttttagcttacaagctacttaaagaaagccaatctaaacgacttgttcttaggtgtaagattttgaagactaagcaaatttctttccatgaaacaacattgtttttgtttagcgatatgccattaagtcgtttatgttctaattaatctggttttgtaggctggtttccatgcaaaacgtattccagcagttagcagtttacaggagcatatagttaaatcaacaccggcaagatatagtagtacacaggcatgtttggaaaatgacatttctggaactgataataaagggtaatttctgttttactttcctaccactggatctctttttttgcattccttgtttatggacagtcattgctaaatgacatggcatttattcatgagtattactcgtcatatgtggaatatacttcactcatttgacataaaagctgcacgtacaagcgtaagaagaattttactaaaacaatgtatctccacttttgcaggttcaagggtcatgatatgttggcacccttcactgctgggtggcaaagtactgatgtggatcctttaattatagagaagtctgaggttagatttatgtctacttttgctgtctaacttaagagaagtttatatatctttcgtgatcaacttttacatttcgacatagggatcccacgtatatgacatgcaagggaggaagtatcttgatactctagctgg

SEQ ID NO: 6 - DNA, 529 bp, Solanum tuberosum
accagcttagattctttaaagaaattgcagaattaaatggaggcaaataagtctatggtgaagttggttgctttcttgataatttttgcatcatgctttcaatctctcactgctcaagatttggaaatcgaagttagtgatggcttaaatgtcttgcaactacatgatgtgtctcagtcattttgtccaggtgtgacgaaagaaagttggccagaacttctagggacaccagctaagtttgcaaagcaaataattcagaaggaaaatccaaaattaacaaatgttgaaactctactgaatggttctgcttttacagaagatttgagatgcaatagagttcgtctttttgttaatttattggacattgttgtacaaactcccaaagttggttaaacaaaattaattcatgttatatatatgtatctagcctccagaaaaataaattggagttgtaatatggttaatgcttccactatatttggtgataaataaacgtggctttttaatattaaaaaaaaaaaaaaaaaaa

SEQ ID NO: 7 - DNA, 2035 bp, Solanum tuberosum
ccgatatttgatttgcaatttagcaacgaattgattcgaaggatcatatcaaatggctaagatttcttgtcttattggatccaccgtcaaagcagctatcaccgcccaggctcctttccatgcaaaacgtattccagcagttagcagtttacaggagcatatagttaaatcaacaccggcaagatatagtagtacacaggcatgtttggaaaatgacatttctggaactgataataaagggttcaagggtcatgatatgttggcacccttcactgctgggtggcaaagtactgatgtggatcctttaattatagagaagtctgagggatcccacgtatatgacatgcaagggaggaagtatcttgatactctagctggtttgtggtgcacagcactaggggggaacgagcctcgcctggttgatgctgccactaagcaattaaacacattgccattttaccattcattttggaaccgtacaacaaaaccttctttggatcttgcgaaggagcttctggatatgtttactgcaaagaaaatggcaaaagcttttttcaccaatagtggatcagaagccaatgatacccaggtgaagctggtttggtattataacaatgctcttggaaggccaaacaaaaagaaatttatagctcgagcaaaagcatatcatggttcaactcttatttctgccagtctcactggtcttcctgcattacatcaaaattttgatcttcctgctccatttgttcttcacaccgactgtcctcattattggcgttatcatctgccaggtgagacagaggaggagttctctaccagattggctaaaaatttggaagatcttatcctcaaagaggggcctgaaacaatagctgctttcattgctgaaccagtcatgggggcaggaggtgtcatacctcctccagctacctattttgataagattcaagctgtagtgaagaaatatgacattcttttcattgcggatgaggtgatctgtgcctttgggaggcttggaacaatgtttggctctgacatgtataacatcaaacctgatcttgtctccttagcaaaggctctttcttctgcatatatgccaattggagctgtccttgtaagccctgaagtttctgatgtaattcattctcaaagcaataaacttggttccttttcccatggattcacttattctgggcatcctgttgcatgcgcggtggcattggaagctattaaaatctacaaggagcgaaatatggttgagagagtaaatacaatatccccaaagtttcaagaaggtctgaaggagttttctgacagtcccattatcggagagattaggggaattggtttgatccttgccacagagtttgcgaataacaaatctcctaatgatcctttccctcctgaatggggtgttggtgcatattttggagcacaatgtcagaagaatggcatgttggtacgtgttgctggtgataccatcatgatgtctcctccatttgtagttactccagaagaacttgacgagttgattagcatctatgggaaagcattgagggaaactgaaaagagagtagaagaactcaagtctcagaagtgatattagttgacagcacaagcttgacgatgacgaaaaaaacaaaaacaaattcaagcacaataaaataaaaaaatcaaatgtgttggatattctgtaaatgtccagaatgaagtaatgagtataatttttagtccaagttgctcctcttctctttcattttacatgcagtatagtttcaccagttcacttattgatgaagatgtctatccccttaaccagttgtcacccaagattaatgcattttaccaaaaaatcgaatttattaatctatgttcttgtaattaattgagttttttttatgttcgagtttgtacgttaatgcacatttctcctataaagtcttttctgtcaataatattttcttaaaagtaatcatgttgtatttgggattcaaataaaaatgaatgctcgccaaacaaaaaaaaaaaaaaaaaa

SEQ ID NO: 8 - DNA, 22 bp, Artificial Sequence
  Description of Artificial Sequence Primer
gtaatacgactcactatagggc

SEQ ID NO: 9 - DNA, 19 bp, Artificial Sequence
  Description of Artificial Sequence Primer
actatagggcacgcgtggt

SEQ ID NO: 10 - DNA, 26 bp, Artificial Sequence
  Description of Artificial Sequence Primer
gaaagcaaccaacttcaccatagact

SEQ ID NO: 11 - DNA, 38 bp, Artificial Sequence
  Description of Artificial Sequence Primer
cttcaccatagacttatttgcctccatttaattctgca

SEQ ID NO: 12 - DNA, 26 bp, Artificial Sequence
  Description of Artificial Sequence Primer
ccagctagagtatcaagatacttcct

SEQ ID NO: 13 - DNA, 28 bp, Artificial Sequence
  Description of Artificial Sequence Primer
cgttcccccctagtgctgtgcaccacaa

SEQ ID NO: 14 - DNA, 27 bp, Artificial Sequence
  Description of Artificial Sequence Primer
gcttagtggcagcatcaaccaggcgag